(19) United States
(12) Patent Application Publication
(10) Pub. No.: US 2011/0126601 A1
(43) Pub. Date: Jun. 2, 2011

(54) ECOTECHNICAL INSTALLATION AND METHOD FOR THE PRODUCTION OF CULTIVATION SUBSTRATES, SOIL AMENDMENTS, AND ORGANIC FERTILIZERS HAVING PROPERTIES OF ANTHROPOGENIC TERRA PRETA SOIL

(76) Inventor: Joachim Böttcher, Hengstbacherhof (DE)

(21) Appl. No.: 12/997,808

(22) PCT Filed: Jun. 12, 2009
(86) PCT No.: PCT/EP2009/004228

§ 371 Date: Jan. 25, 2011

(30) Foreign Application Priority Data
Jun. 13, 2008 (DE) 10 2008 028 144.1
Jun. 18, 2008 (DE) 10 2008 028 907.8

(51) Int. Cl. C05F 11/08; C12M 3/06

(52) U.S. Cl. 71/10; 435/290.1; 435/290.4

(57) ABSTRACT

The invention relates to an ecotechnical installation for producing cultivation substrates, soil amendments, and organic fertilizers having properties of anthropogenic terra preta soil as well as a method for producing cultivation substrates, soil amendments, or organic fertilizers having properties of anthropogenic terra preta soil. The invention further relates to the use of said method or installation for utilizing unpolluted organic biomasses, biowaste, dirty water and sewage, mineral substances for generating current and/or heat and/or producing cultivation substrates having properties of anthropogenic terra preta soil. The invention finally relates to a circulation installation allowing a sustainable material flow management in order to utilize unpolluted organic biomasses, biowaste, mineral substances, etc. for generating current and heat and producing industrial water and cultivation substrates, soil amendments, or organic fertilizers.

### TECHNICAL FIELD

The present invention relates to an ecotechnical installation for producing cultivation substrates and soil amendments and organic fertilisers having properties of anthropogenic terra preta soil and to a method for producing cultivation substrates or soil amendments or organic fertilisers having properties of anthropogenic terra preta soil. The invention further relates to the use of the method or the installation for the recycling of unpolluted organic biomasses, biowaste, dirty water, sewage and mineral substances to provide electric power, heat and/or cultivation substrates having properties of anthropogenic terra preta soil. Finally, the invention also relates to a circulating installation within the meaning of sustainable material flow management for recycling unpolluted organic biomasses, biowaste, mineral materials, etc. to provide electric power, heat, useful water and cultivation substrates or soil amendment or organic fertilisers.

### PRIOR ART

The term “terra preta soil” or “terra preta do indio” (Portuguese for “black earth”) denotes an anthropogenic black earth which archaeologists have discovered in the region of old remains of settlements in the Amazon region. It consists of a mixture of organic waste, bones, clay fragments, charcoal, etc. and was hand-produced by a special method more than 2,000 years ago.

Owing to its high fertility, the terra preta soil is described as the “black gold of the Amazon region”.

The cultivation of this earth enabled Brazilian natives to bring in rich harvests despite having low-nutrient soil. Although the Amazonian rainforests have a lush green face, the vegetation is disappointing in that it rarely produces humus and few nutrients in the soil.

The soils (oxisol) are instead low in nutrients and unsuitable, in particular for agriculture. Only the natural circulation system of the rainforest maintains complex material management which provides for plants and animals. Once the rainforest has disappeared, however, the soils rapidly become completely infertile. The term Amazonian is therefore synonymous with “wet desert” which can only offer food to a few humans.

It was all the more surprising what the conqueror Franscisco de Oreliana allegedly saw in 1542 when he discovered the Amazon region, large settlements and masses of long-haired fighters bristling with weapons. The Spaniard thought they were women, hence the female name “Amazons”. For a long time, historians assumed that de Oreliana had discovered freely. After him, no one else saw many people in the Amazon region as it was unclear how they should feed themselves. Nowadays, archaeologists assume, however, that de Oreliana was right. The soil form “terra preta do indio” produced by the Indians had created fertile land and therefore the basis for greater population densities in the Amazon region. According to the latest scientific findings, it can be assumed that the Amazon region was settled by civilisations with large settlements for more than a thousand years and accommodated 5 to 6 million people.

When the civilisations disappeared, so did their knowledge of terra preta soil. Archaeologists did not discover the black gold of the Amazon region until the end of the 20^{th} century. Its fertility has remained until now and is unique throughout the world.

In contrast to the early civilisations in the Amazon region, there is still terra preta soil in numerous locations, it is so fertile that it is sold, for example, as potting compost. The civilizations which first created the terra preta soil may have perished from diseases brought by the Europeans.

Various research works have shown that terra preta soil is more than a mere blend of known input materials. It has in the meantime also been scientifically demonstrated what extraordinary potential lies in these soils. However, artificial methods for producing this valuable soil are as yet virtually unknown.

Further positive effects of terra preta soil with respect to substrates and horticulturally or agriculturally used soils reside in the high water retention capacity, cation exchange capacity, buffering capacity for plant nutrients and in the good aeration. These are all properties which make a crucial contribution to the quality of cultivation substrates and soils.

The inventor succeeded in identifying and reproducing the essential processes involved in terra preta soil formation. The result is a cultivation substrate which, even under European conditions, soon deploys action mechanisms corresponding to those of the original “terra preta do indio”.

Former methods known in the prior art do not make it possible to produce a cultivation substrate or soil amendment having the properties of anthropogenic terra preta soil. Such methods are described, for example, in U.S. Pat. No. 6,200,475 B, EP 0 168 556 A, WO 01/00543 A and EP 0 676 385 A.

### BRIEF DESCRIPTION OF THE INVENTION

It is therefore an object of the present invention to provide an installation and a method with which it is possible to carry out ecologically and economically efficient recycling of unpolluted organic biomasses, biowaste, dirty water, sewage and mineral substances, etc. to provide electric power, heat, useful water and terra preta soil as a cultivation substrate and soil amendment and organic fertilisers, and to provide a cultivation substrate or soil amendment or organic fertiliser having the properties of anthropogenic terra preta soil.

This object is achieved by an ecotechnical installation having the features of claim 1 and by a method for producing cultivation substrates or soil amendments or organic fertilisers having properties of anthropogenic terra preta soil according to claim 18.

The terra preta soil produced according to the invention is a nutrient-rich humus substrate with good soil-forming properties. The nutrients are stably bound in the substrate and therefore are not washed out. They are made available via specific action mechanisms in accordance with plant requirements, so the plants are completely provided with nutrients. This is effected on the one hand via very good physical properties (for example cation exchange capacity and nutrient storage capacity) and on the other hand via particularly balanced population of microorganisms which complement one another in their effect.

In contrast to all formerly known substrates and soil types, moreover, the terra preta soil according to the invention has excellent soil-forming properties, such as the formation of humus. If, for example, a horticulturally or agriculturally used soil is worked up with terra preta soil, the positive properties of the terra preta soil are transferred to the treated soil. The soils treated in this way maintain their effectiveness, for example nutrient storage and humus formation, for many years.

The ecotechnical installation according to the invention for producing cultivation substrates and soil amendments and organic fertilisers having properties of anthropogenic terra preta soil comprises a plurality of treatment stages for unpolluted organic biomasses, biowaste, dirty water, sewage, mineral substances etc., which will be described hereinafter.

A first stage is intended for the case where liquid organic biomasses such as fermentation residues, liquid manure, slops, dirty water, sewage, organic drainage water, etc. are treated and processed in the installation according to the invention. This first stage comprises at least one replacement filter of which the basic structure consists of organic biomass and/or mineral materials and/or inert carbon with a large pore volume and/or a mixture thereof. The liquid organic biomass is conveyed across this filter.

A second stage is intended for the case where the organic biomasses to be treated require hygienisation or contain germinatable seeds, and for the case where the products to be produced such as cultivation substrate, soil amendment and/or organic fertiliser are to be hygienised and freed from germinatable seeds. This second stage consists of at least one intensive rotting during which hygienisation and destruction of germinatable seeds are carried out by increasing the temperature. The increase in temperature also leads to dewatering of the mixture by evaporation.

In a third stage, the unpolluted solid and liquid organic biomasses, biowaste, dirty water, sewage, mineral substances, etc. are subjected to an anaerobic fermentation process. The anaerobic fermentation, in particular the anaerobic lactic acid fermentation, is an essential step in the production of the cultivation substrate or soil amendment or organic fertiliser according to the invention. Anaerobic lactic acid fermentation is preferred.

In a further step, the fermented biomass is subjected to drying and optionally pH neutralisation. Further aggregates may be added to adapt the product properties to the desired step of use.

The ecotechnical installation according to the invention preferably consists of individual windrows, filling vessels, containers and/or boxes, in which the individual method steps are carried out.

### DETAILED DESCRIPTION OF THE INVENTION

Both liquid and solid organic biomasses are processed in the installation according to the invention. Suitable liquid biomasses, include, for example, fermentation residues from a biogas installation and/or liquid manure from animal husbandry and/or dirty water and sewage for the production of terra preta soil, as these substances contain large amounts of plant nutrients. In a preferred embodiment, these liquid input materials are initially pre-sieved in a mechanical separation stage, for example curved sieve, drum sieve, etc. and separated into a solid phase and a liquid phase. The solid phase contains predominantly fibrous parts and coarser particulate constituents. The solid phase can now optionally be further processed separately with addition of pyrogenic carbon and optionally other aggregates to form a soil amendment or organic fertiliser having the properties of anthropogenic terra preta soil and/or may be introduced into the further processing stages of terra preta soil production such as intensive rotting and/or fermentation stage and/or drying stage, while avoiding the replacement filter stage.

The liquid phase formed after separation consists predominantly of smaller organic particles, humic acids, organic and inorganic nutrients and water. This liquid phase is now treated in the replacement filter installation, the liquid being distributed uniformly across the solid organic biomasses and/or mineral materials and/or pyrogenic carbons and/or mixture thereof used in the replacement filter.

The replacement filter preferably consists of from 5 to 30% by volume of inert carbon with a large pore volume or a substitute thereof, from 1 to 30% by volume of mineral constituents and from 40 to 94% by volume of organic biomass.

Filtering, adsorption, absorption and/or biological-chemical conversion of the particulate organic constituents and of the plant nutrients from the liquid biomass takes place in the replacement filter stage. In the process the particulate organic constituents and plant nutrients from the liquid biomass are bound to the solid organic biomasses and/or mineral materials and/or inert carbon with a large pore volume and/or mixture thereof used in the replacement filter.

The type and composition of the end product can vary according to the addition of further aggregates to the liquid biomasses and/or the replacement filter material, so that either a cultivation substrate, soil amendment or organic fertiliser can be produced. For example, magnesium chloride (MgCl₂) can be added to the liquid biomass before and during the treatment in the replacement filter stage, so that larger stable crystalline magnesium ammonium phosphate molecules (MAP) which are retained very well in the replacement filter and produce a good plant fertiliser are formed with the phosphorus and ammonium contained in the liquid.

In a further preferred embodiment, a plurality of replacement filter stages can follow one another in succession, the filter stages consisting of solid organic biomasses and/or mineral materials and/or inert carbon with a large pore volume and/or a mixture thereof and being permeated in succession by the liquid biomass. The filtering, absorption and adsorption capacity of the replacement filter installation are thus increased.

The liquid biomass is applied to the replacement filter, preferably using distributing systems which ensure a uniform distribution on the filter surface such as distributing pipes with openings or movable distributing cars having distributing pipes with openings.

The loading of the replacement filter with liquid biomasses takes place at least once and/or more frequently and is dependent on the filtering, absorption and adsorption capacity of the solid filter materials used. After achieving the filtering, absorption and adsorption capacity of a replacement filter, the loaded material is removed and brought into the subsequent treatment stages such as intensive rotting and/or fermentation stage and/or drying stage, and/or the subsequent treatment stages are set into operation. If there are different filter materials in the replacement filter stages, attention may be paid to the fact, during introduction into the following treatment stages, that a suitable mixing ratio is obtained therein. After removal of the loaded filter material, the replacement filter is refilled with fresh filter material.

In a further preferred embodiment, the liquid biomasses may be applied directly to the replacement filter without preceding separation.

Suitable solid biomasses include any unpolluted organic biomasses with a liquid content of less than 80%, such as tree and shrub cuttings, green waste, straw, harvest residues, press cakes, dregs, solid dung, biowaste, compost, faeces, etc. These biomasses may initially be used as filter material in the replacement filter installation and/or may equally be introduced into the subsequent treatment stages. In order to produce the cultivation substrate or soil amendment or organic fertiliser according to the invention, however, at least the fermentation stage must be passed through.

If the solid biomasses contain relatively large, coarse, caked and/or clumped constituents, they may be shredded, size-reduced and/or sieved prior to further processing in the described treatment stages. If the solid biomasses contain impurities such as glass, plastics material, metal, etc., they may be sorted, sieved, air-separated, etc. prior to further processing in the described treatment stages in order to remove these impurities. However, impurities can also be removed after passing through the treatment stages.

A suitable inert carbon with a large pore volume can be produced within the installation by a suitable technique even for the process. Suitable techniques for producing the inert carbon with a large pore volume may be various methods such as pyrolysis or hydrothermal carbonisation. Pyrolysis is preferably suitable for drier ligin-containing biomasses, whereas hydrothermal carbonisation is suitable for damp, soft biomasses. The appropriate technique for producing the inert carbon with a large pore volume should be selected according to the specific site and is highly dependent on the biomasses to be processed.

In a preferred embodiment, tree and shrub cuttings and green waste are shredded and sieved. Whereas the fine sieve fraction may be introduced directly into the treatment stages such as replacement filter and/or intensive rotting and/or fermentation stage and/or drying stage, the coarse sieve fraction is treated in a pyrolysis installation. Pyrogenic carbon and pyrolysis gas are formed during the pyrolysis process in the absence of air at a temperature of approximately 500° C. Whereas the pyrogenic carbon is introduced as an inert carbon with a large pore volume into the treatment stages for the production of cultivation substrate and/or soil amendment and/or organic fertiliser, the pyrolysis gas may be oxidised in a predominantly or completely flameless manner, for example by the FLOX method. During flameless oxidation, peak temperatures in the flame are avoided and thermal nitrogen oxide formation (NOₓ) is almost completely suppressed, even in the case of high preheating of air, as a result of a high exhaust gas/air premix. A reduction in emissions is achieved by reducing the peak temperature of firing. The resultant pyrolysis gas may however also be recycled elsewhere, for example through cogeneration systems, gas heaters, gas cookers, etc.

FLOX methods, cogeneration systems and gas heaters yield thermal energy which may be employed within the treatment stages, for example to optimise the processes, and/or is used for heating purposes within the production installation. The FLOX method may be additionally equipped with a Stirling motor and then yields electrical energy. The cogeneration system also yields electrical energy.

In a further preferred embodiment, damp soft biomass is introduced into a hydrothermal carbonisation installation. In this case, the biomass is introduced with liquid into a hermetically sealed pressure vessel and is exposed to a temperature greater than 100° C. An exothermic reaction which briefly liberates enormous energy in the form of pressure and heat occurs in the process. The thermal energy may in turn be used within the treatment stages, for example to optimise the processes, and/or may be used for heating purposes within the production installation and/or may be converted into electrical energy. An inert carbon with a large pore volume as well as residual fluid remain in the pressure vessel. Separation of liquid and solid may subsequently take place, the inert carbon then being introduced into the treatment stages.

In a further preferred embodiment, the inert carbon including the residual liquid may be treated via the replacement filter stage where solid/liquid separation again takes place and the carbon remains in the filter material and is subsequently brought to the further treatment stages.

The replacement filter, the intensive rotting means and/or the fermentation stage are preferably constructed as boxes and/or containers and/or windrows, preferably planar windrows or triangular windrows. Filtration, intensive rotting, fermentation and/or drying can be carried out batchwise in the same box, container, planar windrow and/or triangular windrow. In other words, the same box, container, planar windrow or triangular windrow is used for all method steps. Alternatively, filtration, intensive rotting, fermentation and/or drying may be carried out in separate boxes, containers, planar windrows and triangular windrows.

A plurality of replacement filters, intensive rotting means, fermentation stages and optionally drying windrows are provided in a large installation so that a high yield is achieved with the cultivation substrates and/or soil amendments and/or organic fertilisers produced in this way.

As dewatering is to take place in the individual boxes, containers and/or windrows, the floor plate is preferably provided with drainage orifices or, in a further preferred embodiment, has a V-shaped configuration. Depending on the embodiment, the liquids are preferably collected in a tank below the drainage orifices or they open into a central dewatering channel. This dewatering channel leads predominantly to a collecting shaft or a storage shaft for liquid. The liquid guided via the dewatering system to the collecting shaft may also be fed to the replacement filter or other treatment stages for a plurality of cycles for nutrient elimination. Liquid which is substantially freed of nutrients collects in the collecting shaft and/or in the collecting shafts.

In a preferred embodiment, the dewatering devices and/or dewatering channels simultaneously serve to aerate the process located above. For an optimised production run, it is therefore provided that the boxes, containers and/or windrows are equipped with adjustable aeration/deaeration systems and/or dewatering systems. In order to ensure adjustability, slide controls are provided in the individual boxes, containers and/or windrows at the beginning and end of the dewatering section.

For further optimisation of the production process, for example for intensive rotting and/or fermentation, the boxes, containers and/or windrows may be equipped with heatable floors and/or heating tubes for heating and temperature control. An additional buffer store is preferably provided as a heat store for the heating tubes, in which buffer store the required thermal energy may be buffered. In a preferred embodiment, the required thermal energy may be obtained during the above-described pyrolysis process and/or during hydrothermal carbonisation.

In a similarly preferred embodiment, the thermal energy may be obtained via solar thermal systems. In a similarly preferred embodiment, the thermal energy may be obtained via a biogas installation and/or a cogeneration system. In order to use the synergistic effects and sustainable recycling, the heat sources should, where possible, be located within the installation and/or in the immediate vicinity of the installation and/or should be generated from material flows existing in the environment and/or at least from renewable energies.

The intensive rotting phase is used for hygienisation and/or destruction of germinatable seeds and/or water reduction by evaporation. Preferably, the organic biomasses and/or mineral materials and/or inert carbon with a large pore volume and/or a mixture thereof are stacked into a conical pointed heap and/or into a triangular windrow in order to carry out the rotting phase. This is effected by hand and/or using suitable tools such as a conveyor belt and/or wheelbarrow and/or triangular windrow converter. By piling into a pointed heap and/or a triangular windrow, a surface area which is as large as possible for the supply of air and evaporation is achieved. The pointed heap and/or the triangular windrow may be turned several times or mixed to optimise the rotting process, with the result that fresh air is applied to the oxygen-loving microorganisms predominantly participating during the rotting process. In the case of relatively large installations, turning and/or mixing of the windrows is preferably carried out using wheelbarrows and/or triangular windrow converters.

In a preferred embodiment, the pointed heaps and/or triangular windrows are additionally aerated via the floor plates and/or aeration channels. This is preferably carried out using fans which either introduce external air into the floor plates and/or aeration channels and/or introduce air from the replacement filter stage and/or intensive rotting stage and/or fermentation stage and/or drying stage into the floor plates and/or aeration channels.

The following synergistic effects take place during the introduction of air from said treatment stages: outgassed ammonia and CO₂ from the treatment stages are introduced with the air via the floor plates and/or aeration channels into the mixture where some of them are converted back into ammonium and non-gaseous carbon compounds by biochemical processes. The losses of nitrogen from the substrate are thus much reduced and less CO₂ is carried into the atmosphere.

The temperature during intensive rotting is preferably greater than 60° C., more preferably up to 70° C. A reduction of germs, in particular salmonella and pathogenic germs as well as a destruction of germinatable seeds take place at these temperatures. An increase in temperature may be promoted by addition of rot accelerators and/or by addition of fresh biomass. Fresh biomass may be generated, for example, from silage in the winter months. Intensive rotting lasts between three and fourteen days. Intensive rotting may be carried out before or after the fermentation process.

Specific microorganisms and/or fungi are preferably introduced into the mixture to initiate the fermentation process. In a preferred variant, lactic acid bacteria are added to initiate an anaerobic lactic acid fermentation process in the mixture. The introduction of microorganisms and/or fungi preferably takes place in liquid form by spraying and/or blending.

If the fermentation process is carried out in boxes and/or containers in a preferred variant, the lids of the boxes and/or containers are hermetically sealed before carrying out the fermentation process. If the fermentation process takes place in windrows in a further preferred variant, the windrow is pressed relatively flat and compact and covered in an airtight manner, preferably with a sheet. All aeration systems are closed so that anaerobic conditions are ensured.

During fermentation, the pH decreases, among other things due to the activity of the lactic acid bacteria. Fermentation is preferably carried out under acidic conditions, preferably at a pH of less than or equal to 4. The temperature during fermentation is preferably between 30 and 40° C. The duration of fermentation is preferably between one and three weeks, more preferably one to two weeks. Excessively high temperatures should be avoided as they may be detrimental to the microorganisms and thus slow down the process.

Moisture reduction and neutralisation of the fermented biomass takes place in the subsequent drying phase. The biomass is preferably loosened and/or stacked into a pointed heap and/or into a triangular windrow, also in the drying phase. The pH may be regulated and/or the quality of the products may be adapted to the requirements and/or purpose by the addition of neutralising substances such as lime, zeolite, bentonite, clay, sand, lava and/or rock flour. In addition, aeration may be carried out by addition of air (or oxygen), as has already been described for the intensive rotting process. If ammonia and/or CO₂ from the preliminary stages are present in the incoming air, these may in turn be converted to ammonium and stable carbon compounds in the mixture.

A cultivation substrate and/or a soil amendment and/or an organic fertiliser, which have a high nutrient and water storage capacity and correspond to the properties of the anthropogenic terra preta soil are obtained at the end of the process.

It is preferably provided with the installation according to the invention that the windrows or rotting means are enclosed by a housing, preferably a greenhouse. The housing provides for solar heating and thermal energy storage as well as temperature maintenance. In addition, the housing offers protection from weathering (for example from rain and frost) and protection from emissions (the resultant gases remain in the system and are preferably incorporated into the end products in a value-adding manner). It is preferably provided that the air is drawn off in the upper region of the housing and is blown into boxes and/or containers and/or windrows again from below via fans and via aeration systems. The housing is therefore preferably equipped with deaeration systems.

In a preferred embodiment, the roofs of the housing are equipped in part with photovoltaic modules for power generation and/or with solar thermal modules for heat generation. This is a further valuable synergistic effect within the meaning of sustainable recycling.

In a further embodiment, the installation according to the invention additionally comprises a soil filter at the outer edge of the housing and/or in the physical vicinity of the installation. The soil filter is preferably constructed as a raised bed. In a further preferred embodiment, the soil filter is constructed as an earth basin or with stemming. The soil filter is preferably planted with perennial crops such as miscanthus, bamboo, grasses, fruit, berries, etc. which add value. The soil filter contains aeration and dewatering drainage means at the bottom. The aeration and dewatering drainage means is preferably connected to the deaeration system of the housing. Spent and/or excess air can therefore be introduced from the treatment stages in the housing into the aeration and dewatering drainage means of the soil filter. Air flows upwards through the raised bed owing to an excess pressure in the pipe, and this leads, on the one hand, to growth-promoting aeration of the soil filter and, on the other hand, to biological cleaning of the air similarly to a biofilter. The floor filler material itself preferably consists of the terra preta soil cultivation substrate produced. Bacteria in the cultivation substrate can biologically clean the escaping gases.

In a further preferred embodiment, the soil filter is simultaneously used to treat excess and/or contaminated liquids from the production process. For this purpose, the excess and/or contaminated liquid can be applied to the soil filter using distributing devices, pipes, etc. During permeation of the soil filter, the liquid is filtered and/or biologically purified by the microorganisms present. The purified liquid is collected and discharged via the aeration and dewatering drainage means in the soil filter. The liquid is preferably reused as useful water in the production process and/or used for watering purposes, for example energy crop plantations. Additional synergies are thus obtained within the meaning of sustainable recycling. At the same time, the loading of the liquids on the soil filter prevents drying out, as known from normal biofilters. In addition, plant growth is optimised and the yields of crops considerably increased by the good watering of the crops in the soil filter with liquid that sometimes contains nutrients and by the good aeration. In a further preferred embodiment, the soil filter may also be integrated within the housing, so that plant growth can be increased again over temperatures which are more continuous.

In a further aspect, the present invention relates to a device and a method for producing cultivation substrates and/or soil amendments and/or organic fertilisers having properties of anthropogenic terra preta soil, in which no liquid biomass is filtered via a replacement filter. In this case, the first method step consists directly of intensive rotting and fermentation during which, for example, anaerobic lactic fermentation is carried out in the presence of microorganisms. Intensive rotting may be carried out before or after fermentation. A replacement filter is not then required as no liquid biomass is filtered. Solid, finely sieved organic biomass is preferably blended directly with inert carbon having a large pore volume, subjected to intensive rotting and fermented for approximately one to two or three weeks. The biomass is subsequently dried and neutralised in a drying windrow preferably for approximately one week, i.e. the acidic ph (approximately 4) prevailing in the fermentation due to lactic acid fermentation is raised to a neutral level.

The products, cultivation substrate, soil activator and/or organic fertiliser may be optimised and adapted to the desired use using various additional measures such as sieving, addition of aggregates and/or organic plant nutrients from the treatment process and/or another origin, natural minerals, etc.

In addition, the products may be packaged and/or bagged manually and/or mechanically. Scientific investigations have shown that the cultivation substrates or soil amendments produced via the installation or method according to the invention have considerable product advantages:

1. improved plant growth and reduction of failure rates in new planting; reduction of water consumption;

2. lower labour costs for watering;

3. reduction of fertiliser costs by up to 80%;

4. long useful life owing to low nutrient leaching;

5. simple application, introduction into existing cultivations is also possible.

6. Sustainable improvement to soils and their properties by build-up of humus, etc.

The terms “cultivation substrate” and “soil amendment” and “organic fertiliser” used here cover all substrates, earths, organic fertilisers, soil enhancers, soil activators which are suitable for the improvement of soils, use as plant carriers in closed systems (pots, troughs, barrels, etc.), fertilising, roof planting, interior planting, plant substrate, young plant cultivation, potting compost, tree nursery compost, vine nurseries, etc. The products may be brought onto the market in loose form as bulk material, in packaged form as bagged goods, big packs or the like.

The possible applications of the terra preta soils produced according to the invention are wide ranging. The humus substrate may be used as a plant and cultivation substrate for professional horticulture or as potting compost for hobby gardening.

Possible applications in the field of intensive horticulture (greenhouses, open land), of horticulture and agriculture (soil improvement, roof planting, etc.) are also envisaged.

Further beneficial applications are found in agriculture for improving intensively used or degraded soils.

The potential and possible applications of terra preta soils for global environmental protection and for a global improvement in the supply of food will also be indicated at this point.

Arid regions may be made fertile again by the application of terra preta soil owing to the excellent water storage and humus forming properties (stemming of global desertification).

The vegetation thus established will have a positive effect on the global climate if used extensively and the consequences of the climate change will be stemmed. A further aspect is envisaged in the stable binding of climate-damaging carbon dioxide in the soil in the form of long-lasting humus (CO₂ reduction).

If terra preta soil technology is adopted extensively in agriculture, there will be greater efficiency per unit area with simultaneous sparing of the environment and reduction of negative environmental effects by agriculture. As a result, more healthy foods could be produced worldwide and at the same time biomass, for example for energy utilisation.

Currently discussed competition over area in agriculture between food production and biomass production could be reduced by using terra preta technology. In a terra preta installation configured according to the invention, an industrial biogas installation for producing renewable energy is to be erected for the fermentation of 50,000 tonnes per year of organic residues such as food and plant residues.

The innovative techniques of the invention allow the fermentation residues to be worked up completely into a marketable, high-quality, humus substrate and therefore represent an intelligent solution to the problem of disposal.

The ecotechnical installation according to the invention for producing cultivation substrates, soil amendments and organic fertilisers having properties of anthropogenic terra preta soil may be a constituent of a complete system which is constructed as a circulating installation. In addition to a terra preta production plant, the complete installation may comprise a biogas installation and a subsequent energy crop plantation (for example miscanthus). Owing to the coupling of biogas installation with a terra preta soil production plant, a closed material circuit is to be achieved with which electric power, heat and a high quality humus substrate are produced from biogenic waste.

The liquid fermentation residues formed in the biogas installation are treated via predominantly near-natural technology, the replacement filter installation according to the invention playing a decisive role.

In the installation according to the invention, the fermentation residues are preferably initially conveyed via a mechanical separating installation, where separation into solids and liquids takes place.

Whereas the solids separated in the process pass directly into the terra preta soil installation for further processing, the liquid constituents are further treated in the replacement filter installation. The remaining solids and nutrients from the liquid are filtered out in the process and are now also available for the production of terra preta soil.

After the treatment in the replacement filter installation for the retention of particulate organic substances and nutrients, the liquid can still be conveyed via a plant clarifier (soil filter) where the organic contamination is still further reduced and the liquid treated in this way can also be buffered for prolonged periods.

From the aspect of sustainable recycling and the closure of material cycles, the innovative model of an extensive energy crop plantation with China grass (miscanthus) is integrated in an installation according to the invention, the residual nutrients contained in the excess liquids being utilised on the one hand and an optimum supply of water to the plants being achieved on the other hand. Biomass yields of over 300 tonnes of fresh mass per year are anticipated.

The harvested product miscanthus can be used in a versatile manner, for example as wood chips for biomass heating, for pellet production or as structural and fibrous material for building material production. However, the introduction of this biomass into the production process for optimising terra preta soil is anticipated as a focus.

The biogas installation according to the invention can produce energy by fermentation of organic residues.

It is intended to use 50,000 tonnes per year of organic waste such as food residues, plant residues, fat separators, etc. Cogeneration systems (BHKW) with a total electric power of 1.5 MW have to be installed in order to recycle the biogas produced. Yields are generated by introducing the electric power into the public mains and by selling the thermal energy to an adjoining wood pellet producing factory.

Production of the terra preta soil-like humus substrate is basically possible wherever organic biomasses are formed, produced or present, for example in biogas installations, agriculture, horticulture, land management, processing industry, viticulture, distilleries, dairies, animal husbandry, composting plants, waste water treatment plants, etc.

### BRIEF DESCRIPTION OF THE DRAWINGS

The invention is described in more detail in the following drawings, in which:

FIG. 1 is a plan view of the construction of an ecotechnical installation according to the invention for producing cultivation substrates and/or soil amendments and/or organic fertilisers having properties of anthropogenic terra preta soil;

FIG. 2 is a side view of the installation shown in FIG. 1.

### WAYS OF CARRYING OUT THE INVENTION AND INDUSTRIAL USABILITY

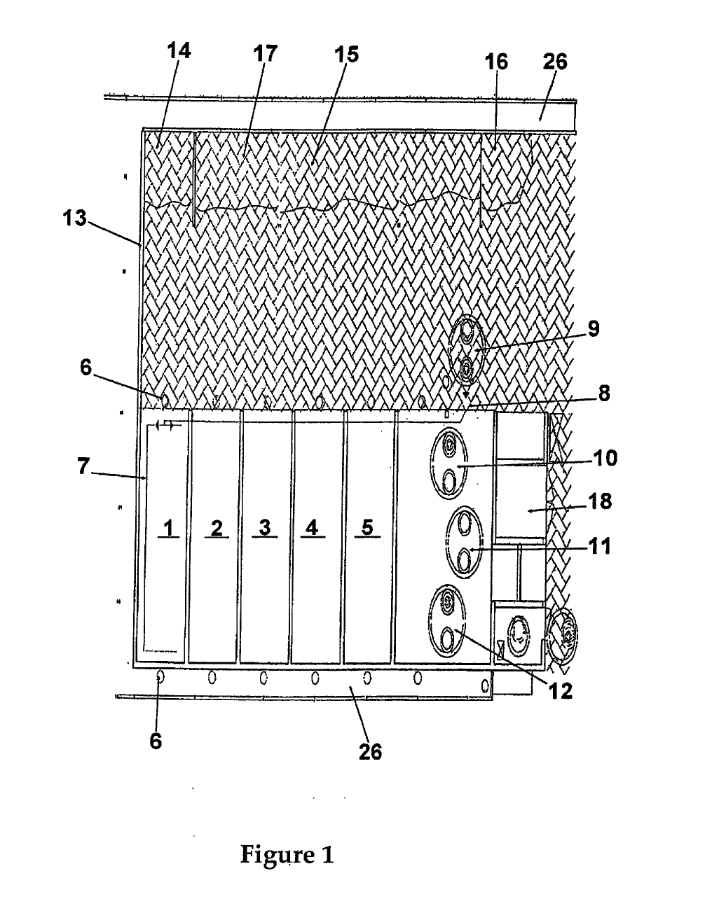

FIG. 1 is a horizontal projection in a plan view of an embodiment of an ecotechnical installation according to the invention. This ground plan shows the essential constituents for producing cultivation substrates or soil amendments or organic fertilisers having properties of anthropogenic terra preta soil. The installation can be constructed as an individual plant or as a constituent of a circulating installation (together with a biogas installation, composting installation, processing installation for organic biomasses and an energy crop plantation).

Liquid fermentation residues and/or liquid manures and/or dirty water and sewage, which are buffered in storage containers, shafts, tanks, etc. are used, for example, as starting substrates for the method according to the invention. Solid biomasses, for example in the form of green waste, solid dung, straw, wood chips, chopped wood, compost, etc. which are buffered in reserve areas may also be used. This solid biomass, for example unsieved green waste, is initially size-reduced (shredded) and sieved to form a fine and a coarse sieve fraction. The finely sieved organic biomass (for example sieved green waste) is stored in a reserve region 17.

In addition to the biomasses, further aggregates with substrate-improving properties may also be used (for example rocks, earth, soils, lava, pumice, basalt, clay, loam, bentonite, lime, sandstone grit, etc.). The addition of mineral or organic nutrients is also advantageous; organic fibrous products (for example coconut fibres, wood chips, turf, etc.) may also be added. Aggregates of this type may be stored in separate reserve regions. A reserve region of this type for lava is shown as reserve region 16. The further constituents required for the process such as inert carbon (for example charcoal, lignite, pyrolysis products, HTC products) are also stored separately. Premixing for the replacement filter 1 can take place in a mixing zone 14. The liquid biomass is applied to the replacement filter 1 via a distributing device 24.

The actual terra preta soil production method takes place in the windrows in zones 1, 2, 3, 4 and 5. Blending of the finely sieved organic biomass with the inert carbon, preferably charcoal, produced in the pyrolysis process takes place in the replacement filter 1, shown as an individual windrow in this case. The pyrogenic carbon (charcoal) is produced in a pyrolysis device 18). The pyrolysis device 18 comprises a FLOX burner, in which pyrolysis can take place at temperatures of approximately 500° C. Combustion takes place completely or predominantly flamelessly, so the level of emissions can be kept to a minimum.

In addition, liquid organic biomasses such as fermentation residues, dirty water, sewage, liquid manure, etc. which are buffered in a storage shaft 9 can be added. The liquid is conveyed via a dewatering channel 7 to a collecting shaft 10 or back into the storage shaft 9. The individual dewatering systems 7 of the windrows 1, 2, 3, 4, 5 are connected to the storage shaft 9 or collecting shaft 10 via corresponding pipes 8. The individual windrows are dewatered and/or aerated via closable slider orifices 6.

The floor plates of the windrows have a V-shaped configuration, forming a slight gradient to the central dewatering channel 7 for the discharge of liquid. Organic particles are filtered out via complex material structures in the mixture of the replacement filter and nutrients are absorbed and taken up.

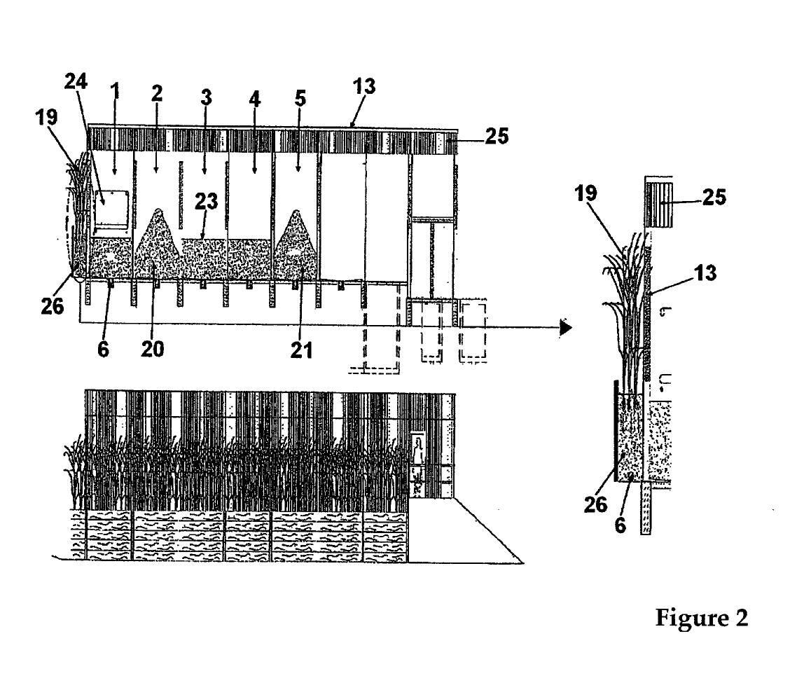

For an optimum result, 10% by volume of charcoal, 10% by volume of mineral constituents (for example lava, clay, pumice) and 80% by volume of organic biomass (for example fine green waste) are mixed together during replacement filtration and loaded for approximately one week with liquid nutrient-rich biomasses such as fermentation residues, liquid manure, dirty water, sewage, etc. This is followed by an intensive rotting phase in the windrow 2 for approximately three to seven days. The intensive rotting may however also or additionally be carried out after fermentation. Hygienisation and destruction of germinatable seeds takes place in the intensive rotting. New biomass (for example green waste, mineral constituents, silage) can optionally be added to the intensive rotting. The temperatures are more than 60° C. and preferably 70 ° C. the biomass is preferably piled up into a pointed heap 20, 21 and/or a triangular windrow, in order to provide a large replacement area for air (cf. FIG. 2).

The fermentation phase with anaerobic lactic acid fermentation is carried out in the two fermenters 3 and 4. Lactic acid fermentation takes place at a temperature of preferably 30 to 40° C. for a period of approximately one to 3 weeks. In order to ensure anaerobic conditions, the windrow is covered in an airtight manner by a sheet. The pH decreases to below 4 during the fermentation process. All drainage orifices 6 are closed during the fermentation phase so that no oxygen can impair the anaerobic conditions. Drying and neutralisation in the drying windrow 5 take place after fermentation. Drying lasts approximately one week and may be optimised by feeding air via the aeration and deaeration device. Neutralisation is carried out by adding neutralising substances such as lime, zeolite, bentonite or rock flour.

In order to maintain the temperature, the individual windrows 1, 2, 3, 4, 5 are provided with floor heating and/or heating tubes. In this way, the individual windrows 1, 2, 3, 4, 5 can be used in a versatile manner, i.e. each individual windrow can be used as a replacement filter, intensive rotting means, fermenter or drying windrow. A batch method is conceivable, so that the entire procedure is carried out in the same windrow. If five windrows are used, five parallel procedures would therefore be possible for producing the cultivation substrate or soil amendment or organic fertiliser according to the invention.

In the embodiment shown, the floor heating and/or heating tubes for temperature control are supplied via a buffer store 11. The cistern 12 shown thereunder is used for the storage of useful water (for example rainwater from gutters of the housing 13). The entire installation is enclosed by a housing 13. Outside the housing 13 is a raised bed as a soil filter 26. The raised bed or the soil filter 26 in turn communicates with the individual windrows 1, 2, 3, 4, 5 via drainage orifices 6. The drainage orifices 6 may be opened or closed independently of one another via the individual sliding controls.

FIG. 2 is a side view of the installation according to the invention. The biomass is introduced flat in the replacement filter 1 and in the two fermenters 3, 4. The biomass is poured into a pointed heap 20 or 21 in the intensive rotting means 2 and the drying windrow 5. A maximum surface area is thus ensured for air exchange and evaporation of water. The above-mentioned raised bed as a soil filter 26 is located outside the housing 13. Miscanthus and/or bamboo and/or other crops 19 are planted therein in a terra preta soil cultivation substrate. The raised bed treats liquids from the production process and converts gases from the outgoing air (for example ammonia) into stable nitrogen compounds (for example ammonium ions). A deaeration system 25 is located at the inner ridge of the housing 13.

1-40. (canceled)

41. Ecotechnical installation for producing cultivation substrates and/or soil amendments and/or organic fertilisers having properties of anthropogenic terra preta soil, comprising
at least one fermenter (3, 4) in which unpolluted solid and liquid organic biomasses, biowaste, dirty water, sewage and mineral substances are subjected to an anaerobic fermentation process, in particular anaerobic lactic acid fermentation, at least inert carbon with a large pore volume or a substitute thereof such as lava, zeolite, bentonite, expanded clay and/or other porous material being contained in the fermenter (3, 4), characterised by
at least one biological replacement filter (1) which precedes the fermenter (3, 4) for liquid organic biomasses such as fermentation residues, liquid manure, slops, dirty water and sewage which, in its basic structure, consists of organic biomass and/or mineral materials and/or inert carbon with a large pore volume such as charcoal or HTC carbon and/or a mixture thereof and across which the liquid organic biomasses are conveyed,
at least one intensive rotting means (20) for hygienisation and for destroying germinatable seeds via an increase in temperature, and
at least one drying windrow (5) for drying and neutralising the fermented biomass.

42. Installation according to claim 41, characterised in that the installation additionally comprises a mechanical separator in which the liquid biomass is pre-sieved and separated into a solid phase and a liquid phase.

43. Installation according to any one of the preceding claims, characterised in that the replacement filter consists of from 5 to 30% by volume of inert carbon with a large pore volume or a substitute thereof, from 1 to 30% by volume of mineral constituents and from 40 to 94% by volume of organic biomass.

44. Installation according to any one of the preceding claims, characterised in that the replacement filter (1), the intensive rotting means (2) and/or the fermenter (3, 4) are constructed as boxes, containers or windrows.

45. Installation according to any one of the preceding claims, characterised in that the base plate of the windrows has drainage orifices with a tank located thereunder or has a V-shaped configuration and opens into a central dewatering channel (7) which leads to a collecting shaft (10) or a storage shaft (9) for liquid.

46. Installation according to any one of the preceding claims, characterised in that the boxes, containers or windrows are equipped with adjustable aeration/deaeration systems, watering/dewatering systems and/or with floor heating and/or heating tubes for heating and temperature control.

47. Installation according to any one of the preceding claims, characterised in that the treatment stages are enclosed by a housing (13), preferably a greenhouse, which is equipped with a deaeration system (25).

48. Installation according to any one of the preceding claims, characterised in that a soil filter (26) is located at the outer edge of the housing (13) and/or in the vicinity of the installation, wherein the soil filter (26) may be a raised bed or an earth basin or may be stemmed and is planted with perennial crops such as miscanthus, bamboo, grass, fruit, berries and has an aeration and dewatering drainage means (6) at the bottom.

49. Installation according to any one of the preceding claims, characterised in that the installation additionally comprises a pyrolysis installation for producing the inert carbon or a hydrothermal carbonisation (HTC) installation.

50. Method for producing cultivation substrates or soil amendments or organic fertilisers having properties of anthropogenic terra preta soil in which unpolluted solid and liquid organic biomasses, biowaste, dirty water, sewage and mineral substances are subjected to at least one anaerobic fermentation stage, in particular anaerobic lactic acid fermentation, wherein at least inert carbon with a large pore volume or a substitute thereof such as lava, zeolite, bentonite, expanded clay and/or other porous materials is contained in the fermentation stage, characterised in that, in a biological filtration stage preceding the fermentation stage, liquid organic biomasses such as fermentation residues, liquid manure, slops, dirty water and sewage are conveyed across a biological replacement filter which, in its basic structure, consists of organic biomass and/or mineral materials and/or inert carbon with a large pore volume and/or a mixture thereof, in a further stage, intensive rotting is carried out for hygienisation and destruction of germinatable seeds by increasing the temperature, and the fermented biomass is dried and neutralised in a further stage.

51. Method according to claim 50, characterised in that the liquid biomass is subjected to a mechanical separation stage during which the biomass is separated into a solid phase and a liquid phase.

52. Method according to any one of the preceding claims, characterised in that aggregates such as rock, earth, soil, lava, pumice, basalt, clay, loam, bentonite, lime, sandstone grit and/or magnesium chloride and/or fibrous organic products are added to the liquid biomass.

53. Method according to any one of the preceding claims, characterised in that a plurality of replacement filter stages follow one another in succession, the filter stages consisting of solid organic biomasses and/or mineral materials and/or inert carbon and being permeated in succession by the liquid biomass.

54. Method according to any one of the preceding claims, characterised in that the intensive rotting is carried out before or after the fermentation process.

55. Method according to any one of the preceding claims, characterised in that lactic acid bacteria are added to the biomass to be fermented in the fermenter in order to carry out anaerobic lactic acid fermentation.

56. Method according to any one of the preceding claims, characterised in that neutralisation in the drying stage is carried out by addition of lime, zeolite, bentonite and/or rock flour.

57. Method according to any one of the preceding claims, characterised in that filtration, intensive rotting, fermentation and/or drying are carried out batchwise in the same box, container or windrow or in separate boxes, containers or windrows.

58. Method according to any one of the preceding claims, characterised in that the boxes, containers or windrows are aerated and/or dewatered.

59. Use of an installation according to any one of claims 41 to 49 or of a method according to any one of claims 50 to 58 for producing a cultivation substrate and/or soil amendment and/or organic fertiliser.

60. Use of an installation according to any one of claims 41 to 49 or of a method according to any one of claims 50 to 58 for recycling unpolluted organic biomasses, biowaste, dirty water, sewage and mineral substances to provide electric power, heat and/or cultivation substrates and/or soil amendments and/or organic fertilisers having properties of anthropogenic terra preta soil.

61. Circulating installation within the meaning of sustainable material flow management for the recycling of unpolluted organic biomasses, biowaste, mineral substances, etc. to provide electric power, heat, useful water and cultivation substrates or soil amendments or organic fertilisers, comprising a biogas installation and/or composting installation and/or processing installation for organic biomasses and/or an energy crop plantation, characterised in that the circulating installation further comprises an installation for producing cultivation substrates and/or soil amendments and/or organic fertilisers having properties of anthropogenic terra preta soil according to any one of claims 41 to 49.
